(19) United States
(12) Patent Application Publication
(10) Pub. No.: US 2022/0347185 A1
(43) Pub. Date: Nov. 3, 2022

(54) IBOGAINE FORMULATIONS

(71) Applicant: CAAMTECH, INC., Issaquah, WA (US)

(72) Inventor: Andrew R. CHADEAYNE, Issaquah, WA (US)

(21) Appl. No.: 17/620,855

(22) PCT Filed: Jun. 24, 2020
(86) PCT No.: PCT/US2020/039312

§ 371 Date: Dec. 20, 2021

Related U.S. Application Data
(60) Provisional application No. 62/865,514, filed on Jun. 24, 2019.

(51) Int. Cl. A61K 31/55

(52) U.S. Cl. CPC A61K 31/55

(57) ABSTRACT

Disclosed herein are new compositions and methods comprising a combination of active ingredients that includes at least one ibogaine derivative. Several selected examples of non-naturally occurring ibogaine derivative formulations are also disclosed. The ibogaine derivatives are combined into formulations via human ingenuity to arrive at compositions and formulations that are not found in nature. These formulations have different physical properties from those found in nature and also provide different pharmacological properties. In many cases, the below disclosed combinations provide different clinical effects when administered to a subject. The disclosed compositions are useful in many contexts, including treating addictions, such as alcohol, nicotine, and opiate addictions.

### CROSS-REFERENCE TO RELATED APPLICATION

This application claims priority to U.S. Application No. 62/865,514, filed Jun. 24, 2019, which is incorporated herein by reference.

### TECHNICAL FIELD

This disclosure relates to non-natural forms of natural medicines, in particular new formulations of ibogaine derivatives.

### BACKGROUND

Addiction affects millions of people worldwide. The abuse of legal and illegal drugs, tobacco, and alcohol take their toll on individuals, families, communities, and society. The addiction problem extends to dysfunctional behaviors involving gambling, food, sex, exercise, shopping, etc. According to the Addiction Center, in 2011 there were 20.6 million people in the United States over the age of 12 with an addiction (excluding tobacco).¹ Mental health conditions often accompany addiction.² This is often seen with substance abuse disorders and gambling addictions. Almost half of the substance abusers in the United States have mental disorders.³

Psychology Today says addiction is “a condition in which a person engages in the use of a substance or in a behavior for which the rewarding effects provide a compelling incentive to repeatedly pursue the behavior despite detrimental consequences.”² Addictive substances and behaviors share the neurological feature of activating the reward and reinforcement pathways in the brain. These pathways often use the neurotransmitter dopamine.

There are an estimated 38 million adult smokers in the United States.⁴ This is about 15.5% of the population. The World Health Organization (WHO) estimates there are 1.1 billion people worldwide who smoke.⁵ In addition, tobacco is considered the leading cause of death, illness, and impoverishment in the world. The WHO reports more than 7 million people worldwide die from tobacco-related illnesses each year.

According to the National Institute on Drug Abuse, opioid overdoses account for more than 130 deaths every day in the United States.⁶ In 2017, over 47,000 Americans died of an opiate overdose. Using data from 2007-2013, the National Institute on Drug Abuse estimated that tobacco, illicit drugs, and alcohol combined to cost the United States over $740 billion annually.⁷ These costs are related to lost work productivity, crime, and healthcare expenses. The costs continue to skyrocket. The estimated cost of America's drug epidemic alone in 2017 exceeded $1.0 trillion.⁸

Historically, addiction was often thought of as a pleasure-seeking activity undertaken by people with a weak personal constitution and insufficient self-control. However, the current concept of addiction is changing based on scientific findings. It is now understood that a main driver of addictive behaviors is to escape physical and/or emotional discomfort.⁹ This is called self-medication. It is interesting to note that the term addiction did not appear in the diagnostic manual of the American Psychiatric Association (Diagnostic and Statistical Manual of Mental Disorders, the DSM) until the recent 5^{th} edition update.

Addiction experts now recognize that substance abuse disorders and non-substance abuse disorders have a lot in common. They have grouped them into the Syndrome Model of Addiction⁹ This model suggests there is one addiction which is associated with multiple expressions. The scientific basis of this model includes imaging data that shows the brain responds similarly to different pleasurable experiences whether ingesting alcohol, shopping, sex, or gambling. In addition, studies have shown that some people are genetically predisposed to addiction but not to a specific type of addiction. Overall, it appears the object of a person's addiction (a substance or behavior) is less important than experts originally thought.

Ibogaine (CAS 83-74-9) is a psychedelic tryptamine alkaloid first isolated in 1901 from the root bark of the Tabernanthe iboga shrub (also called iboga) of Central Africa¹⁰ and the root bark of a shrub in the genus Tabernaemontana found in the Congo.¹¹ Since that time, ibogaine has been isolated from several plant species particularly those in the family Apocynaceae to which T. iboga belongs.

The crystalline structure of ibogaine was determined in 1960.¹² It was first synthesized in 1966.¹³ Ibogaine has two separate chiral centers and four stereoisomers but they are difficult to resolve.¹⁴ Plants containing ibogaine as well as extracts and preparations made from them include many other active and inactive molecules including several different ibogaine derivatives.

Historically, iboga plants have been used for centuries in the religious rituals of people living the western part of Central Africa in countries such as the Republic of the Congo, Gabon, and Cameroon.¹⁵ The ceremonial cocktails contained a mixture of the naturally-occurring molecules found in the plant. Harrison G. Pope of the Harvard Botanical Museum theorized in 1969 that humans learned about the effects of iboga by watching the behavior of animals.¹⁶ First-hand accounts describe native animals like porcupines, boars, and gorillas digging up and eating iboga roots. After ingesting the roots they would enter “a wild frenzy.” These indigenous people also use less potent versions of ibogaine cocktails to treat fatigue, hunger, and thirst.

In the 1930s, French pharmacists developed ibogaine as a drug which was sold as a stimulant under the trade name Lambaréné.¹⁷ Here, ibogaine was taken in its pure form without all the other active molecules and modulators of those molecules that are present in the plant. The drug was taken off the market in the 1960s when France declared ibogaine illegal.

Howard Lotsof was the first person to realize the potential of ibogaine to treat substance addictions.¹⁸ He was a heroin addict when he tried ibogaine in 1962 with several of his friends who were also addicts. They were surprised to find it caused marked reductions in their cravings and withdrawal symptoms. All of them quit using heroin as a result. Lotsof went on to become a scientist and dedicated his life to studying ibogaine for treating addiction.

Ibogaine exhibits relatively low potency at target sites. Studies in rats suggest high doses may cause adverse effects. In minor treatment, high doses of 5.0 mg/kg body weight are used and up to 30 mg/kg body weight for intense poly-substance addiction management.

In 1988, Dzoljic et al. were the first to publish on the ability of ibogaine to relieve withdrawal from narcotics addiction.¹⁹ Maisonneuve et al. elucidated the pharmacological interactions between ibogaine and morphine in 1991.²⁰ After this, several other researchers showed ibogaine's ability to reduce or interrupt the self-administration of opiates in rats and mice and alter their behaviors.²¹⁻²⁵ Additional study results showed ibogaine was more effective in multiple administrations over time than from a single dose.^{21,22}

Recent review papers and meta-analyses have concluded that ibogaine is effective for treating substance addiction and warrants further investigation.²⁶⁻²⁸ Several current studies have found ibogaine effective for treating opioid addiction.²⁹⁻³²

There is also experimental evidence indicating that ibogaine and ibogaine derivatives help in relieving depressive symptoms. For example, in 2000, Mash et al. found that ibogaine significantly decreased depressive symptoms in humans.³³ A mouse study published in 2006 by da Silva et al. reported that the ibogaine derivative montanine had not only antidepressive effects in mice, but was also anxiolytic and anticonvulsive.³⁴ In a study done in 2020, Rodriguez et al. observed that a single dose of ibogaine or its metabolite noribogaine caused antidepressant effects in rats.³⁵

The known ibogaine derivatives exhibit substantial variability in their efficacy and side effects. And, naturally-occurring combinations of ibogaine derivatives (like extracts and other combinations used in traditional plant medicine) produce different effects vis-à-vis other combinations and/or individual ibogaine derivatives.

At the time of this disclosure, ibogaine derivatives were known to exist in two forms, either A) a single active ingredient or B) within a full spectrum plant extract or preparation.

There is an unmet need for formulated products providing specific combinations of purified ibogaine derivatives wherein each of the purified ibogaine derivatives is separated from the other plant material with which it coexisted in nature.

### SUMMARY

Disclosed herein are new formulations, compositions and methods comprising a combination of active ingredients that includes at least one ibogaine derivative. Several selected examples of non-naturally occurring ibogaine derivative formulations are also disclosed. The ibogaine derivatives are combined into formulations via human ingenuity to arrive at compositions and formulations that are not found in nature. These formulations have different physical properties from those found in nature and also provide different pharmacological properties. In many cases, the below disclosed combinations provide different clinical effects when administered to a subject. The disclosed compositions are useful in many contexts, including treating addictions, such as alcohol, nicotine, and opiate addictions.

Disclosed are formulations comprising a combination comprising, consisting essentially of, or consisting of at least first purified ibogaine derivative and a second ibogaine derivative. The disclosure also provides a method of treating addiction in a human person in need of treatment, comprising the step of administering a therapeutically effective amount of such a formulation to the person in need of treatment. The disclosure further provides a method of treating depression in a human person in need of treatment, comprising the step of administering a therapeutically effective amount of such a formulation to the person in need of treatment.

### DETAILED DESCRIPTION

Disclosed herein are new methods and compositions comprising at least one ibogaine derivative. Below, several selected examples of non-naturally occurring formulations of ibogaine derivatives are disclosed. In each of the exemplary embodiments below, the ibogaine derivatives are combined into formulations via human ingenuity to arrive at compositions and formulations that are not found in nature. These formulations have different physical properties from what is found in nature and also provide different pharmacological properties. In many cases, the below disclosed combinations provide different clinical effects when administered to a subject. For example, administering a combination of ibogaine and noribogaine provides different pharmacological properties and clinical outcomes vis-a-vis either of the individual purified compounds.

Within the context of this disclosure, the term “ibogaine derivative” means a molecule that shares a similar core structure to the compound ibogaine, for example, compounds (ibogaine derivates) such as dihydroxyibogamine, dihydrocatharanthine, coronaridine, conopharyngine, conoflorine, catharanthine, iboxygaine, iboluteine, ibogamine, ibogaline, ibogaine, epiibogamine, isovoacangine, isovoacristine, kisantin, montanin, noribogaine, tabernanthine, tubotaiwine, voacristine, voacangine, voaluteine, and voacamine have been identified in plants such as Alstonia boonei, Anacampta spp., Aspidiosperma, Callichilia barteri, Catharanthus roseus, Creoceras, Ervatamia yunnanensis, Ervatamia divaricate, Ervatamia orientalis, Ervatamia officinalis, Murraya koenigii, Peschiera echinate, Pesheria fuchsiae folia, Rejoua, Rhazya stricta, Schzozygia, Stemmadenia, Strychnos angustiflora, Tabernanthe iboga, Tabernanthe Africana, Tabernanthe australis, Tabernanthe catharinensis, Tabernaemontana corymbose, Tabernaemontana divaricate, Tabernaemontana eglandulosa, Tabernaemontana elegans, Tabernaemontana heyneana, Tabernaemontana hilariana, Tabernaemontana holstii, Tabernaemontana johnstonii, Tabernaemontana undulata, Tabernanthe pubescens, Trachelospermum jasminoides, Vinca minor, Voacanga Africana, Voacanga schweinfurthii, and Voacanga globosa. Such compounds can be either acquired from naturally occurring sources (e.g., by extracting plant material and separating individual alkaloids) or they can be made synthetically in the laboratory. Within the context of this disclosure, “ibogaine derivative” can refer to a molecule in iboga that is regarded as being derived from ibogaine (i.e., a molecule with a similar chemical structure), although the pathway(s) by which it is made (either in nature or in the lab) is not yet understood.

Within the context of this disclosure “ibogaine derivative” can also mean a non-tryptamine molecule in a plant that produces ibogaine as a natural product, for example a molecule produced within the plant that influences the pharmacology of ibogaine or other tryptamines found in iboga.

Within the context of this disclosure, the term “total ibogaine derivatives” refers to the sum of all ibogaine derivatives. The amount (e.g., mass or moles) of “total ibogaine derivatives” in a particular composition, such as a naturally occurring source material (e.g., iboga) or a composition or formulation can be calculated by summing the ibogaine derivatives. The relative amount or percentage of a particular ibogaine derivative with respect to the total ibogaine derivatives can be calculated by taking the quotient of the particular ibogaine derivative(s) subject to analysis and dividing it by the total ibogaine derivatives present in the sample. For example, in a sample containing 1 mole of ibogaine and 2.5 moles of total ibogaine derivatives, the percentage of ibogaine vis-a-vis the total ibogaine derivative would be (1÷2.5)*100%=40%.

Within the context of this disclosure the term “administering” means delivering a compound (e.g., an active ingredient, such as ibogaine and/or other iboga compounds) to a particular organism, such as a human. Within the context of this disclosure the term “administering” includes oral administration, intraperitoneal administration (IP), intravenous administration (IV), transdermal administration, inhalation administration, nasal administration, anal administration, and vaginal administration, etc. For example, ibogaine via IP and IV routes as described by Rodriguez et al. in 2020 in the journal ACS Chemical Neuroscience.³⁵ Another example of how to administer ibogaine derivatives orally can be found in a 2015 study by Alper et al. in Cardiovascular Toxicology describes orally administering ibogaine to human subjects.³⁶

Determining the correct dosage of ibogaine derivatives can be accomplished by monitoring the concentration of ibogaine in human blood plasma and correlating that concentration with the desired pharmacological or clinical outcome. The blood plasma levels of ibogaine derivatives can be determined by and/or inferred from various methods known in the art. For example, the 2020 study by Rodriguez et al. describes methods of determining the amount of ibogaine and other iboga compounds in the plasma of animals.³⁵

As used herein the phrase “wherein the first purified ibogaine derivative is different from the second ibogaine derivative” means that the ibogaine derivative molecule of the “first purified ibogaine derivative” is structurally different from that of the second ibogaine derivative. Such structural differences, could be determined and demonstrated by a variety of analytical methods known in the chemical arts, including but not limited to chromatography, NMR, x-ray crystallography, etc.

In one embodiment, the compositions disclosed herein comprise one or more ibogaine derivatives chosen from the following: dihydroxyibogamine, dihydrocatharanthine, coronaridine, conopharyngine, conoflorine, catharanthine, iboxygaine, iboluteine, ibogamine, ibogaline, ibogaine, epiibogamine, isovoacangine, isovoacristine, kisantin, montanin, noribogaine, tabernanthine, tubotaiwine, voacristine, voacangine, voaluteine, and voacamine.

The ibogaine derivative dihydroxyibogamine is a compound having the following structural formula:

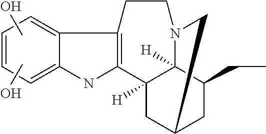

Dihydroxyibogamine is naturally present in many naturally occurring organisms, including Tabernaemontana heyneana, and Tabernanthe iboga. In nature, Dihydroxyibogamine always occurs as a minor component within a mixture of many other molecules.

The ibogaine derivative dihydrocatharanthine is a compound having the following structural formula:

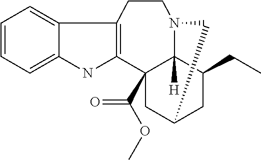 [SMILES: CC[C@H]1C[C@@H]2C[N@@]3CCc4c([nH]c5ccccc45)[C@@](C(=O)OC)(C2)[C@@H]13]

Dihydrocatharanthine is naturally present in many naturally occurring organisms, including Tabernanthe iboga. In nature, Dihydrocatharanthine always occurs as a minor component within a mixture of many other molecules.

The ibogaine derivative coronaridine is a compound having the following structural formula:

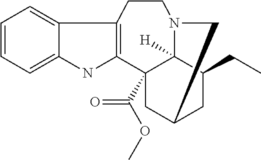 [SMILES: CC[C@H]1C[C@H]2CN3CCc4c([nH]c5ccccc45)[C@](C(=O)OC)(C2)[C@H]13]

Coronaridine is naturally present in many naturally occurring organisms, including Ervatamia spp., Tabernaemontana australis, Tabernaemontana divaricata, Tabernaemontana eglandulosa, Tabernaemontana heyneana, Tabernaemontana hilariana, Tabernaemontana holstii, and Tabernanthe iboga. In nature, Coronaridine always occurs as a minor component within a mixture of many other molecules.

The ibogaine derivative conopharyngine is a compound having the following structural formula:

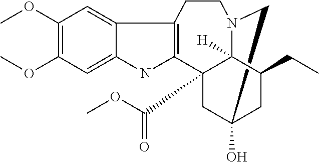 [SMILES: CC[C@H]1C[C@@]2(O)CN3CCc4c([nH]c5cc(OC)c(OC)cc45)[C@](C(=O)OC)(C2)[C@H]13]

Conopharyngine is naturally present in many naturally occurring organisms, including Tabernanthe iboga. In nature, Conopharyngine always occurs as a minor component within a mixture of many other molecules.

The ibogaine derivative conoflorine is a compound having the following structural formula:

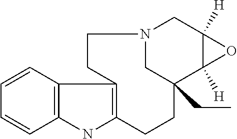 [SMILES: CC[C@]12CCc3[nH]c4ccccc4c3CCN(C[C@H]3O[C@H]31)C2]

Conoflorine is naturally present in many naturally occurring organisms, including Tabernaemontana divaricata, and Tabernanthe iboga. In nature, Conoflorine always occurs as a minor component within a mixture of many other molecules.

The ibogaine derivative catharanthine is a compound having the following structural formula:

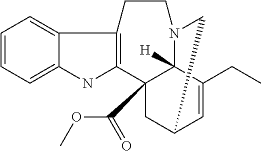 [SMILES: CCC1=C[C@@H]2CN3CCc4c([nH]c5ccccc45)[C@@](C(=O)OC)(C2)[C@@H]13]

Catharanthine is naturally present in many naturally occurring organisms, including Tabernaemontana divaricata, Tabernaemontana hilariana, and Tabernanthe iboga. In nature, Catharanthine always occurs as a minor component within a mixture of many other molecules.

The ibogaine derivative iboxygaine is a compound having the following structural formula:

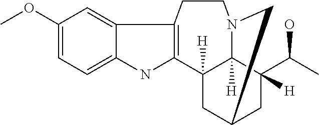 [SMILES: COc1ccc2[nH]c3c(c2c1)CCN1C[C@@H]2C[C@@H]([C@H](C)O)[C@H]1[C@H]3C2]

Iboxygaine is naturally present in many naturally occurring organisms, including Ervatamia orientalis, and Tabernanthe iboga. In nature, Iboxygaine always occurs as a minor component within a mixture of many other molecules.

The ibogaine derivative iboluteine is a compound having the following structural formula:

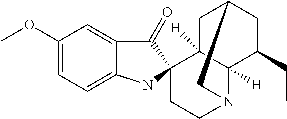 [SMILES: CC[C@@H]1C[C@H]2C[C@H]3[C@@H]1N(CC[C@@]31Nc3ccc(OC)cc3C1=O)C2]

Iboluteine is naturally present in many naturally occurring organisms, including Tabernanthe iboga. In nature, Iboluteine always occurs as a minor component within a mixture of many other molecules.

The ibogaine derivative ibogamine means a compound having the following structural formula:

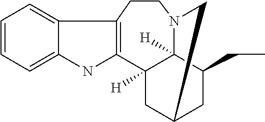 [SMILES: CC[C@H]1C[C@@H]2C[C@H]3c4[nH]c5ccccc5c4CCN(C2)[C@@H]13]

Ibogamine is naturally present in many naturally occurring organisms, including Tabernaemontana australis, Tabernaemontana eglandulosa, Tabernaemontana hilariana, Tabernaemontana johnstonii, and Tabernanthe iboga. In nature, Ibogamine always occurs as a minor component within a mixture of many other molecules.

The ibogaine derivative ibogaline is a compound having the following structural formula:

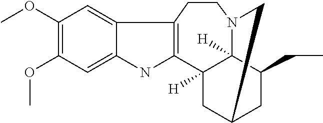 [SMILES: CC[C@H]1C[C@@H]2C[C@H]3c4[nH]c5cc(OC)c(OC)cc5c4CCN(C2)[C@@H]13]

Ibogaline is naturally present in many naturally occurring organisms, including Tabernaemontana australis, and Tabernanthe iboga. In nature, Ibogaline always occurs as a minor component within a mixture of many other molecules.

The ibogaine derivative ibogaine is a compound having the following structural formula:

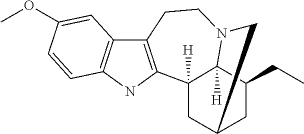 [SMILES: CC[C@H]1C[C@@H]2C[C@H]3c4[nH]c5ccc(OC)cc5c4CCN(C2)[C@@H]13]

Ibogaine is naturally present in many naturally occurring organisms, including Ervatamia orientalis, Tabernaemontana australis, and Tabernanthe iboga. In nature, Ibogaine always occurs as a minor component within a mixture of many other molecules.

The ibogaine derivative epiibogamine is a compound having the following structural formula:

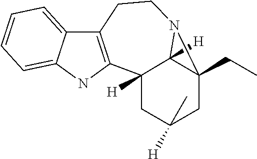

Epiibogamine is naturally present in many naturally occurring organisms, including Tabernanthe iboga. In nature, Epiibogamine always occurs as a minor component within a mixture of many other molecules.

The ibogaine derivative isovoacangine is a compound having the following structural formula:

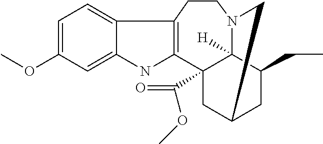 [SMILES: CC[C@H]1C[C@H]2CN3CCc4c([nH]c5cc(OC)ccc45)[C@](C(=O)OC)(C2)[C@H]13]

Isovoacangine is naturally present in many naturally occurring organisms, including Tabernaemontana divaricata, Tabernaemontana hilariana, Tabernaemontana johnstonii, and Tabernanthe iboga. In nature, Isovoacangine always occurs as a minor component within a mixture of many other molecules.

The ibogaine derivative isovoacristine means a compound having the following structural formula:

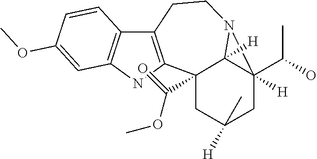

Isovoacristine is naturally present in many naturally occurring organisms, including Tabernanthe iboga. In nature, Isovoacristine always occurs as a minor component within a mixture of many other molecules.

The ibogaine derivative kisantin is a compound having the following structural formula:

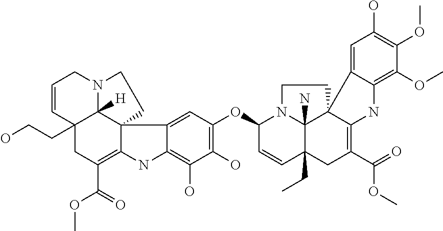

Kisantin is naturally present in many naturally occurring organisms, including Tabernanthe iboga. In nature, Kisantin always occurs as a minor component within a mixture of many other molecules.

The ibogaine derivative montanin is a compound having the following structural formula:

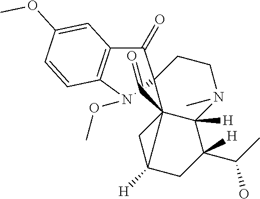

Montanin is naturally present in many naturally occurring organisms, including Tabernanthe iboga. In nature, Montanin always occurs as a minor component within a mixture of many other molecules.

The ibogaine derivative noribogaine is a compound having the following structural formula:

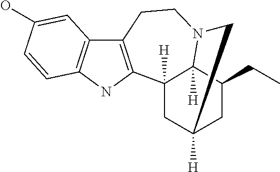 [SMILES: CC[C@H]1C[C@@H]2C[C@H]3c4[nH]c5ccc(O)cc5c4CCN(C2)[C@@H]13]

Noribogaine is naturally present in many naturally occurring organisms, including Tabernanthe iboga. In nature, Noribogaine always occurs as a minor component within a mixture of many other molecules.

The ibogaine derivative tabernanthine is a compound having the following structural formula:

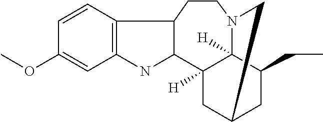 [SMILES: CC[C@H]1C[C@@H]2C[C@H]3C4Nc5cc(OC)ccc5C4CCN(C2)[C@@H]13]

Tabernanthine is naturally present in many naturally occurring organisms, including Tabernaemontana hilariana, and Tabernanthe iboga. In nature, Tabernanthine always occurs as a minor component within a mixture of many other molecules.

The ibogaine derivative tubotaiwine is a compound having the following structural formula:

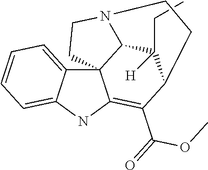 [SMILES: CCC1[C@H]2CCN3CC[C@@]4(C(=C2C(=O)OC)Nc2ccccc24)[C@@H]13]

Tubotaiwine is naturally present in many naturally occurring organisms, including Tabernaemontana divaricata, Tabernaemontana eglandulosa, and Tabernanthe iboga. In nature, Tubotaiwine always occurs as a minor component within a mixture of many other molecules.

The ibogaine derivative voacristine is a compound having the following structural formula:

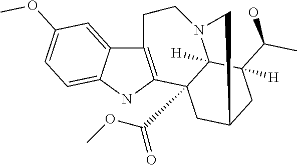 [SMILES: COC(=O)[C@@]12C[C@H]3C[C@H]([C@H](C)O)[C@@H]1N(CCc1c2[nH]c2ccc(OC)cc12)C3]

Voacristine is naturally present in many naturally occurring organisms, including Alstonia scholaris, Ervatamia orientalis, Peschiera buchtieni, Tabernaemontana heyneana, and Tabernanthe iboga. In nature, Voacristine always occurs as a minor component within a mixture of many other molecules.

The ibogaine derivative voacangine is a compound having the following structural formula:

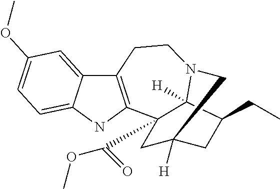 [SMILES: CC[C@H]1C[C@H]2CN3CCc4c([nH]c5ccc(OC)cc45)[C@](C(=O)OC)(C2)[C@H]13]

Voacangine is naturally present in many naturally occurring organisms, including Tabernaemontana australis, Tabernaemontana hilariana, and Tabernanthe iboga. In nature, Voacangine always occurs as a minor component within a mixture of many other molecules.

The ibogaine derivative voaluteine is a compound having the following structural formula:

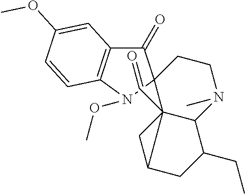

Voaluteine is naturally present in many naturally occurring organisms, including Tabernanthe iboga. In nature, Voaluteine always occurs as a minor component within a mixture of many other molecules.

The ibogaine derivative voacamine is a compound having the following structural formula:

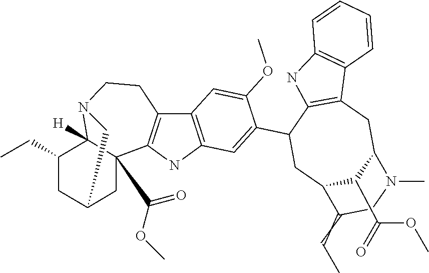 [SMILES: CC=C1CN(C)[C@H]2Cc3c([nH]c4ccccc34)C(c3cc4[nH]c5c(c4cc3OC)CCN3C[C@@H]4C[C@H](CC)[C@H]3[C@@]5(C(=O)OC)C4)C[C@@H]1C2C(=O)OC]

Voacamine is naturally present in many naturally occurring organisms, including Ervatamia orientalis, Voacanga africana, and Voacanga schweinfurthii. In nature, Voacamine always occurs as a minor component within a mixture of many other molecules.

Disclosed herein are methods and compositions utilizing a purposefully chosen combination of two or more individual ibogaine derivatives and optionally an inactive compound, such as an excipient. The total amount of ibogaine derivatives in a formulation may range from 0.01 to 100 wt. %, from 0.1 to 100 wt. %, from 1 to 99 wt. %, from 50 to 90 wt. %, from 5 to 75 wt. %, from 10 to 50 wt. %, from 10 to 25 wt. %, or from 15 to 40 wt. %. A purposefully chosen combination comprises, consists essentially of or consists of a first purified ibogaine derivative and a second ibogaine derivative, wherein the first purified ibogaine derivative is different from the second ibogaine derivative. Exemplary molar ratios of the first purified ibogaine derivative to the second derivative include but are not limited to from 0.1:100 to 100:01, from 1:100 to 100:1, from 1:50 to 50:1, from 1:25 to 25:1, from 1:20 to 20:1, from 1:10 to 10:1, from 1:5 to 5:1, from 1:2 to 2:1 or may be 1:1. Where a purposefully chosen combination contains more than two ibogaine derivatives each ibogaine derivative within the purposefully chosen combination will be present in a molar ratio such as just mentioned with respect to each other ibogaine derivative present. The combination containing more than two ibogaine derivatives may then be considered as a combination or mixture of first and second ibogaine derivatives. For example, a formulation may contain purposefully chosen combination of purified dihydroxyibogamine, and dihydrocatharanthine and additionally coronaridine, each vis-à-vis the other ibogaine derivations within one or more of the molar ratios above. Exemplary formulations containing such purposefully chosen combinations of the disclosure as described herein are listed below.

One formulation comprises a purposefully chosen combination of purified dihydroxyibogamine, and dihydrocatharanthine. One formulation comprises a purposefully chosen combination of purified dihydroxyibogamine, and coronaridine. One formulation comprises a purposefully chosen combination of purified dihydrocatharanthine, and coronaridine. One formulation comprises a purposefully chosen combination of purified dihydroxyibogamine, and conopharyngine. One formulation comprises a purposefully chosen combination of purified dihydrocatharanthine, and conopharyngine. One formulation comprises a purposefully chosen combination of purified coronaridine, and conopharyngine. One formulation comprises a purposefully chosen combination of purified dihydroxyibogamine, and conoflorine. One formulation comprises a purposefully chosen combination of purified dihydrocatharanthine, and conoflorine. One formulation comprises a purposefully chosen combination of purified coronaridine, and conoflorine. One formulation comprises a purposefully chosen combination of purified conopharyngine, and conoflorine. One formulation comprises a purposefully chosen combination of purified dihydroxyibogamine, and catharanthine. One formulation comprises a purposefully chosen combination of purified dihydrocatharanthine, and catharanthine. One formulation comprises a purposefully chosen combination of purified coronaridine, and catharanthine. One formulation comprises a purposefully chosen combination of purified conopharyngine, and catharanthine. One formulation comprises a purposefully chosen combination of purified conoflorine, and catharanthine. One formulation comprises a purposefully chosen combination of purified dihydroxyibogamine, and iboxygaine. One formulation comprises a purposefully chosen combination of purified dihydrocatharanthine, and iboxygaine. One formulation comprises a purposefully chosen combination of purified coronaridine, and iboxygaine. One formulation comprises a purposefully chosen combination of purified conopharyngine, and iboxygaine. One formulation comprises a purposefully chosen combination of purified conoflorine, and iboxygaine. One formulation comprises a purposefully chosen combination of purified catharanthine, and iboxygaine. One formulation comprises a purposefully chosen combination of purified dihydroxyibogamine, and iboluteine. One formulation comprises a purposefully chosen combination of purified dihydrocatharanthine, and iboluteine. One formulation comprises a purposefully chosen combination of purified coronaridine, and iboluteine. One formulation comprises a purposefully chosen combination of purified conopharyngine, and iboluteine. One formulation comprises a purposefully chosen combination of purified conoflorine, and iboluteine. One formulation comprises a purposefully chosen combination of purified catharanthine, and iboluteine. One formulation comprises a purposefully chosen combination of purified iboxygaine, and iboluteine. One formulation comprises a purposefully chosen combination of purified dihydroxyibogamine, and ibogamine. One formulation comprises a purposefully chosen combination of purified dihydrocatharanthine, and ibogamine. One formulation comprises a purposefully chosen combination of purified coronaridine, and ibogamine. One formulation comprises a purposefully chosen combination of purified conopharyngine, and ibogamine. One formulation comprises a purposefully chosen combination of purified conoflorine, and ibogamine. One formulation comprises a purposefully chosen combination of purified catharanthine, and ibogamine. One formulation comprises a purposefully chosen combination of purified iboxygaine, and ibogamine. One formulation comprises a purposefully chosen combination of purified iboluteine, and ibogamine. One formulation comprises a purposefully chosen combination of purified dihydroxyibogamine, and ibogaline. One formulation comprises a purposefully chosen combination of purified dihydrocatharanthine, and ibogaline. One formulation comprises a purposefully chosen combination of purified coronaridine, and ibogaline. One formulation comprises a purposefully chosen combination of purified conopharyngine, and ibogaline. One formulation comprises a purposefully chosen combination of purified conoflorine, and ibogaline. One formulation comprises a purposefully chosen combination of purified catharanthine, and ibogaline. One formulation comprises a purposefully chosen combination of purified iboxygaine, and ibogaline. One formulation comprises a purposefully chosen combination of purified iboluteine, and ibogaline. One formulation comprises a purposefully chosen combination of purified ibogamine, and ibogaline. One formulation comprises a purposefully chosen combination of purified dihydroxyibogamine, and ibogaine. One formulation comprises a purposefully chosen combination of purified dihydrocatharanthine, and ibogaine. One formulation comprises a purposefully chosen combination of purified coronaridine, and ibogaine. One formulation comprises a purposefully chosen combination of purified conopharyngine, and ibogaine. One formulation comprises a purposefully chosen combination of purified conoflorine, and ibogaine. One formulation comprises a purposefully chosen combination of purified catharanthine, and ibogaine. One formulation comprises a purposefully chosen combination of purified iboxygaine, and ibogaine. One formulation comprises a purposefully chosen combination of purified iboluteine, and ibogaine. One formulation comprises a purposefully chosen combination of purified ibogamine, and ibogaine. One formulation comprises a purposefully chosen combination of purified ibogaline, and ibogaine. One formulation comprises a purposefully chosen combination of purified dihydroxyibogamine, and epiibogamine. One formulation comprises a purposefully chosen combination of purified dihydrocatharanthine, and epiibogamine. One formulation comprises a purposefully chosen combination of purified coronaridine, and epiibogamine. One formulation comprises a purposefully chosen combination of purified conopharyngine, and epiibogamine. One formulation comprises a purposefully chosen combination of purified conoflorine, and epiibogamine. One formulation comprises a purposefully chosen combination of purified catharanthine, and epiibogamine. One formulation comprises a purposefully chosen combination of purified iboxygaine, and epiibogamine. One formulation comprises a purposefully chosen combination of purified iboluteine, and epiibogamine. One formulation comprises a purposefully chosen combination of purified ibogamine, and epiibogamine. One formulation comprises a purposefully chosen combination of purified ibogaline, and epiibogamine. One formulation comprises a purposefully chosen combination of purified ibogaine, and epiibogamine. One formulation comprises a purposefully chosen combination of purified dihydroxyibogamine, and isovoacangine. One formulation comprises a purposefully chosen combination of purified dihydrocatharanthine, and isovoacangine. One formulation comprises a purposefully chosen combination of purified coronaridine, and isovoacangine. One formulation comprises a purposefully chosen combination of purified conopharyngine, and isovoacangine. One formulation comprises a purposefully chosen combination of purified conoflorine, and isovoacangine. One formulation comprises a purposefully chosen combination of purified catharanthine, and isovoacangine. One formulation comprises a purposefully chosen combination of purified iboxygaine, and isovoacangine. One formulation comprises a purposefully chosen combination of purified iboluteine, and isovoacangine. One formulation comprises a purposefully chosen combination of purified ibogamine, and isovoacangine. One formulation comprises a purposefully chosen combination of purified ibogaline, and isovoacangine. One formulation comprises a purposefully chosen combination of purified ibogaine, and isovoacangine. One formulation comprises a purposefully chosen combination of purified epiibogamine, and isovoacangine. One formulation comprises a purposefully chosen combination of purified dihydroxyibogamine, and isovoacristine. One formulation comprises a purposefully chosen combination of purified dihydrocatharanthine, and isovoacristine. One formulation comprises a purposefully chosen combination of purified coronaridine, and isovoacristine. One formulation comprises a purposefully chosen combination of purified conopharyngine, and isovoacristine. One formulation comprises a purposefully chosen combination of purified conoflorine, and isovoacristine. One formulation comprises a purposefully chosen combination of purified catharanthine, and isovoacristine. One formulation comprises a purposefully chosen combination of purified iboxygaine, and isovoacristine. One formulation comprises a purposefully chosen combination of purified iboluteine, and isovoacristine. One formulation comprises a purposefully chosen combination of purified ibogamine, and isovoacristine. One formulation comprises a purposefully chosen combination of purified ibogaline, and isovoacristine. One formulation comprises a purposefully chosen combination of purified ibogaine, and isovoacristine. One formulation comprises a purposefully chosen combination of purified epiibogamine, and isovoacristine. One formulation comprises a purposefully chosen combination of purified isovoacangine, and isovoacristine. One formulation comprises a purposefully chosen combination of purified dihydroxyibogamine, and kisantin. One formulation comprises a purposefully chosen combination of purified dihydrocatharanthine, and kisantin. One formulation comprises a purposefully chosen combination of purified coronaridine, and kisantin. One formulation comprises a purposefully chosen combination of purified conopharyngine, and kisantin. One formulation comprises a purposefully chosen combination of purified conoflorine, and kisantin. One formulation comprises a purposefully chosen combination of purified catharanthine, and kisantin. One formulation comprises a purposefully chosen combination of purified iboxygaine, and kisantin. One formulation comprises a purposefully chosen combination of purified iboluteine, and kisantin. One formulation comprises a purposefully chosen combination of purified ibogamine, and kisantin. One formulation comprises a purposefully chosen combination of purified ibogaline, and kisantin. One formulation comprises a purposefully chosen combination of purified ibogaine, and kisantin. One formulation comprises a purposefully chosen combination of purified epiibogamine, and kisantin. One formulation comprises a purposefully chosen combination of purified isovoacangine, and kisantin. One formulation comprises a purposefully chosen combination of purified isovoacristine, and kisantin. One formulation comprises a purposefully chosen combination of purified dihydroxyibogamine, and montanin. One formulation comprises a purposefully chosen combination of purified dihydrocatharanthine, and montanin. One formulation comprises a purposefully chosen combination of purified coronaridine, and montanin. One formulation comprises a purposefully chosen combination of purified conopharyngine, and montanin. One formulation comprises a purposefully chosen combination of purified conoflorine, and montanin. One formulation comprises a purposefully chosen combination of purified catharanthine, and montanin. One formulation comprises a purposefully chosen combination of purified iboxygaine, and montanin. One formulation comprises a purposefully chosen combination of purified iboluteine, and montanin. One formulation comprises a purposefully chosen combination of purified ibogamine, and montanin. One formulation comprises a purposefully chosen combination of purified ibogaline, and montanin. One formulation comprises a purposefully chosen combination of purified ibogaine, and montanin. One formulation comprises a purposefully chosen combination of purified epiibogamine, and montanin. One formulation comprises a purposefully chosen combination of purified isovoacangine, and montanin. One formulation comprises a purposefully chosen combination of purified isovoacristine, and montanin. One formulation comprises a purposefully chosen combination of purified kisantin, and montanin. One formulation comprises a purposefully chosen combination of purified dihydroxyibogamine, and noribogaine. One formulation comprises a purposefully chosen combination of purified dihydrocatharanthine, and noribogaine. One formulation comprises a purposefully chosen combination of purified coronaridine, and noribogaine. One formulation comprises a purposefully chosen combination of purified conopharyngine, and noribogaine. One formulation comprises a purposefully chosen combination of purified conoflorine, and noribogaine. One formulation comprises a purposefully chosen combination of purified catharanthine, and noribogaine. One formulation comprises a purposefully chosen combination of purified iboxygaine, and noribogaine. One formulation comprises a purposefully chosen combination of purified iboluteine, and noribogaine. One formulation comprises a purposefully chosen combination of purified ibogamine, and noribogaine. One formulation comprises a purposefully chosen combination of purified ibogaline, and noribogaine. One formulation comprises a purposefully chosen combination of purified ibogaine, and noribogaine. One formulation comprises a purposefully chosen combination of purified epiibogamine, and noribogaine. One formulation comprises a purposefully chosen combination of purified isovoacangine, and noribogaine. One formulation comprises a purposefully chosen combination of purified isovoacristine, and noribogaine. One formulation comprises a purposefully chosen combination of purified kisantin, and noribogaine. One formulation comprises a purposefully chosen combination of purified montanin, and noribogaine. One formulation comprises a purposefully chosen combination of purified dihydroxyibogamine, and tabernanthine. One formulation comprises a purposefully chosen combination of purified dihydrocatharanthine, and tabernanthine. One formulation comprises a purposefully chosen combination of purified coronaridine, and tabernanthine. One formulation comprises a purposefully chosen combination of purified conopharyngine, and tabernanthine. One formulation comprises a purposefully chosen combination of purified conoflorine, and tabernanthine. One formulation comprises a purposefully chosen combination of purified catharanthine, and tabernanthine. One formulation comprises a purposefully chosen combination of purified iboxygaine, and tabernanthine. One formulation comprises a purposefully chosen combination of purified iboluteine, and tabernanthine. One formulation comprises a purposefully chosen combination of purified ibogamine, and tabernanthine. One formulation comprises a purposefully chosen combination of purified ibogaline, and tabernanthine. One formulation comprises a purposefully chosen combination of purified ibogaine, and tabernanthine. One formulation comprises a purposefully chosen combination of purified epiibogamine, and tabernanthine. One formulation comprises a purposefully chosen combination of purified isovoacangine, and tabernanthine. One formulation comprises a purposefully chosen combination of purified isovoacristine, and tabernanthine. One formulation comprises a purposefully chosen combination of purified kisantin, and tabernanthine. One formulation comprises a purposefully chosen combination of purified montanin, and tabernanthine. One formulation comprises a purposefully chosen combination of purified noribogaine, and tabernanthine. One formulation comprises a purposefully chosen combination of purified dihydroxyibogamine, and tubotaiwine. One formulation comprises a purposefully chosen combination of purified dihydrocatharanthine, and tubotaiwine. One formulation comprises a purposefully chosen combination of purified coronaridine, and tubotaiwine. One formulation comprises a purposefully chosen combination of purified conopharyngine, and tubotaiwine. One formulation comprises a purposefully chosen combination of purified conoflorine, and tubotaiwine. One formulation comprises a purposefully chosen combination of purified catharanthine, and tubotaiwine. One formulation comprises a purposefully chosen combination of purified iboxygaine, and tubotaiwine. One formulation comprises a purposefully chosen combination of purified iboluteine, and tubotaiwine. One formulation comprises a purposefully chosen combination of purified ibogamine, and tubotaiwine. One formulation comprises a purposefully chosen combination of purified ibogaline, and tubotaiwine. One formulation comprises a purposefully chosen combination of purified ibogaine, and tubotaiwine. One formulation comprises a purposefully chosen combination of purified epiibogamine, and tubotaiwine. One formulation comprises a purposefully chosen combination of purified isovoacangine, and tubotaiwine. One formulation comprises a purposefully chosen combination of purified isovoacristine, and tubotaiwine. One formulation comprises a purposefully chosen combination of purified kisantin, and tubotaiwine. One formulation comprises a purposefully chosen combination of purified montanin, and tubotaiwine. One formulation comprises a purposefully chosen combination of purified noribogaine, and tubotaiwine. One formulation comprises a purposefully chosen combination of purified tabernanthine, and tubotaiwine. One formulation comprises a purposefully chosen combination of purified dihydroxyibogamine, and voacristine. One formulation comprises a purposefully chosen combination of purified dihydrocatharanthine, and voacristine. One formulation comprises a purposefully chosen combination of purified coronaridine, and voacristine. One formulation comprises a purposefully chosen combination of purified conopharyngine, and voacristine. One formulation comprises a purposefully chosen combination of purified conoflorine, and voacristine. One formulation comprises a purposefully chosen combination of purified catharanthine, and voacristine. One formulation comprises a purposefully chosen combination of purified iboxygaine, and voacristine. One formulation comprises a purposefully chosen combination of purified iboluteine, and voacristine. One formulation comprises a purposefully chosen combination of purified ibogamine, and voacristine. One formulation comprises a purposefully chosen combination of purified ibogaline, and voacristine. One formulation comprises a purposefully chosen combination of purified ibogaine, and voacristine. One formulation comprises a purposefully chosen combination of purified epiibogamine, and voacristine. One formulation comprises a purposefully chosen combination of purified isovoacangine, and voacristine. One formulation comprises a purposefully chosen combination of purified isovoacristine, and voacristine. One formulation comprises a purposefully chosen combination of purified kisantin, and voacristine. One formulation comprises a purposefully chosen combination of purified montanin, and voacristine. One formulation comprises a purposefully chosen combination of purified noribogaine, and voacristine. One formulation comprises a purposefully chosen combination of purified tabernanthine, and voacristine. One formulation comprises a purposefully chosen combination of purified tubotaiwine, and voacristine. One formulation comprises a purposefully chosen combination of purified dihydroxyibogamine, and voacangine. One formulation comprises a purposefully chosen combination of purified dihydrocatharanthine, and voacangine. One formulation comprises a purposefully chosen combination of purified coronaridine, and voacangine. One formulation comprises a purposefully chosen combination of purified conopharyngine, and voacangine. One formulation comprises a purposefully chosen combination of purified conoflorine, and voacangine. One formulation comprises a purposefully chosen combination of purified catharanthine, and voacangine. One formulation comprises a purposefully chosen combination of purified iboxygaine, and voacangine. One formulation comprises a purposefully chosen combination of purified iboluteine, and voacangine. One formulation comprises a purposefully chosen combination of purified ibogamine, and voacangine. One formulation comprises a purposefully chosen combination of purified ibogaline, and voacangine. One formulation comprises a purposefully chosen combination of purified ibogaine, and voacangine. One formulation comprises a purposefully chosen combination of purified epiibogamine, and voacangine. One formulation comprises a purposefully chosen combination of purified isovoacangine, and voacangine. One formulation comprises a purposefully chosen combination of purified isovoacristine, and voacangine. One formulation comprises a purposefully chosen combination of purified kisantin, and voacangine. One formulation comprises a purposefully chosen combination of purified montanin, and voacangine. One formulation comprises a purposefully chosen combination of purified noribogaine, and voacangine. One formulation comprises a purposefully chosen combination of purified tabernanthine, and voacangine. One formulation comprises a purposefully chosen combination of purified tubotaiwine, and voacangine. One formulation comprises a purposefully chosen combination of purified voacristine, and voacangine. One formulation comprises a purposefully chosen combination of purified dihydroxyibogamine, and voaluteine. One formulation comprises a purposefully chosen combination of purified dihydrocatharanthine, and voaluteine. One formulation comprises a purposefully chosen combination of purified coronaridine, and voaluteine. One formulation comprises a purposefully chosen combination of purified conopharyngine, and voaluteine. One formulation comprises a purposefully chosen combination of purified conoflorine, and voaluteine. One formulation comprises a purposefully chosen combination of purified catharanthine, and voaluteine. One formulation comprises a purposefully chosen combination of purified iboxygaine, and voaluteine. One formulation comprises a purposefully chosen combination of purified iboluteine, and voaluteine. One formulation comprises a purposefully chosen combination of purified ibogamine, and voaluteine. One formulation comprises a purposefully chosen combination of purified ibogaline, and voaluteine. One formulation comprises a purposefully chosen combination of purified ibogaine, and voaluteine. One formulation comprises a purposefully chosen combination of purified epiibogamine, and voaluteine. One formulation comprises a purposefully chosen combination of purified isovoacangine, and voaluteine. One formulation comprises a purposefully chosen combination of purified isovoacristine, and voaluteine. One formulation comprises a purposefully chosen combination of purified kisantin, and voaluteine. One formulation comprises a purposefully chosen combination of purified montanin, and voaluteine. One formulation comprises a purposefully chosen combination of purified noribogaine, and voaluteine. One formulation comprises a purposefully chosen combination of purified tabernanthine, and voaluteine. One formulation comprises a purposefully chosen combination of purified tubotaiwine, and voaluteine. One formulation comprises a purposefully chosen combination of purified voacristine, and voaluteine. One formulation comprises a purposefully chosen combination of purified voacangine, and voaluteine. One formulation comprises a purposefully chosen combination of purified dihydroxyibogamine, and voacamine. One formulation comprises a purposefully chosen combination of purified dihydrocatharanthine, and voacamine. One formulation comprises a purposefully chosen combination of purified coronaridine, and voacamine. One formulation comprises a purposefully chosen combination of purified conopharyngine, and voacamine. One formulation comprises a purposefully chosen combination of purified conoflorine, and voacamine. One formulation comprises a purposefully chosen combination of purified catharanthine, and voacamine. One formulation comprises a purposefully chosen combination of purified iboxygaine, and voacamine. One formulation comprises a purposefully chosen combination of purified iboluteine, and voacamine. One formulation comprises a purposefully chosen combination of purified ibogamine, and voacamine. One formulation comprises a purposefully chosen combination of purified ibogaline, and voacamine. One formulation comprises a purposefully chosen combination of purified ibogaine, and voacamine. One formulation comprises a purposefully chosen combination of purified epiibogamine, and voacamine. One formulation comprises a purposefully chosen combination of purified isovoacangine, and voacamine. One formulation comprises a purposefully chosen combination of purified isovoacristine, and voacamine. One formulation comprises a purposefully chosen combination of purified kisantin, and voacamine. One formulation comprises a purposefully chosen combination of purified montanin, and voacamine. One formulation comprises a purposefully chosen combination of purified noribogaine, and voacamine. One formulation comprises a purposefully chosen combination of purified tabernanthine, and voacamine. One formulation comprises a purposefully chosen combination of purified tubotaiwine, and voacamine. One formulation comprises a purposefully chosen combination of purified voacristine, and voacamine. One formulation comprises a purposefully chosen combination of purified voacangine, and voacamine. One formulation comprises a purposefully chosen combination of purified voaluteine, and voacamine. One formulation comprises a purposefully chosen combination of purified dihydrocatharanthine, and dihydroxyibogamine. One formulation comprises a purposefully chosen combination of purified coronaridine, and dihydroxyibogamine. One formulation comprises a purposefully chosen combination of purified coronaridine, and dihydrocatharanthine. One formulation comprises a purposefully chosen combination of purified conopharyngine, and dihydroxyibogamine. One formulation comprises a purposefully chosen combination of purified conopharyngine, and dihydrocatharanthine. One formulation comprises a purposefully chosen combination of purified conopharyngine, and coronaridine. One formulation comprises a purposefully chosen combination of purified conoflorine, and dihydroxyibogamine. One formulation comprises a purposefully chosen combination of purified conoflorine, and dihydrocatharanthine. One formulation comprises a purposefully chosen combination of purified conoflorine, and coronaridine. One formulation comprises a purposefully chosen combination of purified conoflorine, and conopharyngine. One formulation comprises a purposefully chosen combination of purified catharanthine, and dihydroxyibogamine. One formulation comprises a purposefully chosen combination of purified catharanthine, and dihydrocatharanthine. One formulation comprises a purposefully chosen combination of purified catharanthine, and coronaridine. One formulation comprises a purposefully chosen combination of purified catharanthine, and conopharyngine. One formulation comprises a purposefully chosen combination of purified catharanthine, and conoflorine. One formulation comprises a purposefully chosen combination of purified iboxygaine, and dihydroxyibogamine. One formulation comprises a purposefully chosen combination of purified iboxygaine, and dihydrocatharanthine. One formulation comprises a purposefully chosen combination of purified iboxygaine, and coronaridine. One formulation comprises a purposefully chosen combination of purified iboxygaine, and conopharyngine. One formulation comprises a purposefully chosen combination of purified iboxygaine, and conoflorine. One formulation comprises a purposefully chosen combination of purified iboxygaine, and catharanthine. One formulation comprises a purposefully chosen combination of purified iboluteine, and dihydroxyibogamine. One formulation comprises a purposefully chosen combination of purified iboluteine, and dihydrocatharanthine. One formulation comprises a purposefully chosen combination of purified iboluteine, and coronaridine. One formulation comprises a purposefully chosen combination of purified iboluteine, and conopharyngine. One formulation comprises a purposefully chosen combination of purified iboluteine, and conoflorine. One formulation comprises a purposefully chosen combination of purified iboluteine, and catharanthine. One formulation comprises a purposefully chosen combination of purified iboluteine, and iboxygaine. One formulation comprises a purposefully chosen combination of purified ibogamine, and dihydroxyibogamine. One formulation comprises a purposefully chosen combination of purified ibogamine, and dihydrocatharanthine. One formulation comprises a purposefully chosen combination of purified ibogamine, and coronaridine. One formulation comprises a purposefully chosen combination of purified ibogamine, and conopharyngine. One formulation comprises a purposefully chosen combination of purified ibogamine, and conoflorine. One formulation comprises a purposefully chosen combination of purified ibogamine, and catharanthine. One formulation comprises a purposefully chosen combination of purified ibogamine, and iboxygaine. One formulation comprises a purposefully chosen combination of purified ibogamine, and iboluteine. One formulation comprises a purposefully chosen combination of purified ibogaline, and dihydroxyibogamine. One formulation comprises a purposefully chosen combination of purified ibogaline, and dihydrocatharanthine. One formulation comprises a purposefully chosen combination of purified ibogaline, and coronaridine. One formulation comprises a purposefully chosen combination of purified ibogaline, and conopharyngine. One formulation comprises a purposefully chosen combination of purified ibogaline, and conoflorine. One formulation comprises a purposefully chosen combination of purified ibogaline, and catharanthine. One formulation comprises a purposefully chosen combination of purified ibogaline, and iboxygaine. One formulation comprises a purposefully chosen combination of purified ibogaline, and iboluteine. One formulation comprises a purposefully chosen combination of purified ibogaline, and ibogamine. One formulation comprises a purposefully chosen combination of purified ibogaine, and dihydroxyibogamine. One formulation comprises a purposefully chosen combination of purified ibogaine, and dihydrocatharanthine. One formulation comprises a purposefully chosen combination of purified ibogaine, and coronaridine. One formulation comprises a purposefully chosen combination of purified ibogaine, and conopharyngine. One formulation comprises a purposefully chosen combination of purified ibogaine, and conoflorine. One formulation comprises a purposefully chosen combination of purified ibogaine, and catharanthine. One formulation comprises a purposefully chosen combination of purified ibogaine, and iboxygaine. One formulation comprises a purposefully chosen combination of purified ibogaine, and iboluteine. One formulation comprises a purposefully chosen combination of purified ibogaine, and ibogamine. One formulation comprises a purposefully chosen combination of purified ibogaine, and ibogaline. One formulation comprises a purposefully chosen combination of purified epiibogamine, and dihydroxyibogamine. One formulation comprises a purposefully chosen combination of purified epiibogamine, and dihydrocatharanthine. One formulation comprises a purposefully chosen combination of purified epiibogamine, and coronaridine. One formulation comprises a purposefully chosen combination of purified epiibogamine, and conopharyngine. One formulation comprises a purposefully chosen combination of purified epiibogamine, and conoflorine. One formulation comprises a purposefully chosen combination of purified epiibogamine, and catharanthine. One formulation comprises a purposefully chosen combination of purified epiibogamine, and iboxygaine. One formulation comprises a purposefully chosen combination of purified epiibogamine, and iboluteine. One formulation comprises a purposefully chosen combination of purified epiibogamine, and ibogamine. One formulation comprises a purposefully chosen combination of purified epiibogamine, and ibogaline. One formulation comprises a purposefully chosen combination of purified epiibogamine, and ibogaine. One formulation comprises a purposefully chosen combination of purified isovoacangine, and dihydroxyibogamine. One formulation comprises a purposefully chosen combination of purified isovoacangine, and dihydrocatharanthine. One formulation comprises a purposefully chosen combination of purified isovoacangine, and coronaridine. One formulation comprises a purposefully chosen combination of purified isovoacangine, and conopharyngine. One formulation comprises a purposefully chosen combination of purified isovoacangine, and conoflorine. One formulation comprises a purposefully chosen combination of purified isovoacangine, and catharanthine. One formulation comprises a purposefully chosen combination of purified isovoacangine, and iboxygaine. One formulation comprises a purposefully chosen combination of purified isovoacangine, and iboluteine. One formulation comprises a purposefully chosen combination of purified isovoacangine, and ibogamine. One formulation comprises a purposefully chosen combination of purified isovoacangine, and ibogaline. One formulation comprises a purposefully chosen combination of purified isovoacangine, and ibogaine. One formulation comprises a purposefully chosen combination of purified isovoacangine, and epiibogamine. One formulation comprises a purposefully chosen combination of purified isovoacristine, and dihydroxyibogamine. One formulation comprises a purposefully chosen combination of purified isovoacristine, and dihydrocatharanthine. One formulation comprises a purposefully chosen combination of purified isovoacristine, and coronaridine. One formulation comprises a purposefully chosen combination of purified isovoacristine, and conopharyngine. One formulation comprises a purposefully chosen combination of purified isovoacristine, and conoflorine. One formulation comprises a purposefully chosen combination of purified isovoacristine, and catharanthine. One formulation comprises a purposefully chosen combination of purified isovoacristine, and iboxygaine. One formulation comprises a purposefully chosen combination of purified isovoacristine, and iboluteine. One formulation comprises a purposefully chosen combination of purified isovoacristine, and ibogamine. One formulation comprises a purposefully chosen combination of purified isovoacristine, and ibogaline. One formulation comprises a purposefully chosen combination of purified isovoacristine, and ibogaine. One formulation comprises a purposefully chosen combination of purified isovoacristine, and epiibogamine. One formulation comprises a purposefully chosen combination of purified isovoacristine, and isovoacangine. One formulation comprises a purposefully chosen combination of purified kisantin, and dihydroxyibogamine. One formulation comprises a purposefully chosen combination of purified kisantin, and dihydrocatharanthine. One formulation comprises a purposefully chosen combination of purified kisantin, and coronaridine. One formulation comprises a purposefully chosen combination of purified kisantin, and conopharyngine. One formulation comprises a purposefully chosen combination of purified kisantin, and conoflorine. One formulation comprises a purposefully chosen combination of purified kisantin, and catharanthine. One formulation comprises a purposefully chosen combination of purified kisantin, and iboxygaine. One formulation comprises a purposefully chosen combination of purified kisantin, and iboluteine. One formulation comprises a purposefully chosen combination of purified kisantin, and ibogamine. One formulation comprises a purposefully chosen combination of purified kisantin, and ibogaline. One formulation comprises a purposefully chosen combination of purified kisantin, and ibogaine. One formulation comprises a purposefully chosen combination of purified kisantin, and epiibogamine. One formulation comprises a purposefully chosen combination of purified kisantin, and isovoacangine. One formulation comprises a purposefully chosen combination of purified kisantin, and isovoacristine. One formulation comprises a purposefully chosen combination of purified montanin, and dihydroxyibogamine. One formulation comprises a purposefully chosen combination of purified montanin, and dihydrocatharanthine. One formulation comprises a purposefully chosen combination of purified montanin, and coronaridine. One formulation comprises a purposefully chosen combination of purified montanin, and conopharyngine. One formulation comprises a purposefully chosen combination of purified montanin, and conoflorine. One formulation comprises a purposefully chosen combination of purified montanin, and catharanthine. One formulation comprises a purposefully chosen combination of purified montanin, and iboxygaine. One formulation comprises a purposefully chosen combination of purified montanin, and iboluteine. One formulation comprises a purposefully chosen combination of purified montanin, and ibogamine. One formulation comprises a purposefully chosen combination of purified montanin, and ibogaline. One formulation comprises a purposefully chosen combination of purified montanin, and ibogaine. One formulation comprises a purposefully chosen combination of purified montanin, and epiibogamine. One formulation comprises a purposefully chosen combination of purified montanin, and isovoacangine. One formulation comprises a purposefully chosen combination of purified montanin, and isovoacristine. One formulation comprises a purposefully chosen combination of purified montanin, and kisantin. One formulation comprises a purposefully chosen combination of purified noribogaine, and dihydroxyibogamine. One formulation comprises a purposefully chosen combination of purified noribogaine, and dihydrocatharanthine. One formulation comprises a purposefully chosen combination of purified noribogaine, and coronaridine. One formulation comprises a purposefully chosen combination of purified noribogaine, and conopharyngine. One formulation comprises a purposefully chosen combination of purified noribogaine, and conoflorine. One formulation comprises a purposefully chosen combination of purified noribogaine, and catharanthine. One formulation comprises a purposefully chosen combination of purified noribogaine, and iboxygaine. One formulation comprises a purposefully chosen combination of purified noribogaine, and iboluteine. One formulation comprises a purposefully chosen combination of purified noribogaine, and ibogamine. One formulation comprises a purposefully chosen combination of purified noribogaine, and ibogaline. One formulation comprises a purposefully chosen combination of purified noribogaine, and ibogaine. One formulation comprises a purposefully chosen combination of purified noribogaine, and epiibogamine. One formulation comprises a purposefully chosen combination of purified noribogaine, and isovoacangine. One formulation comprises a purposefully chosen combination of purified noribogaine, and isovoacristine. One formulation comprises a purposefully chosen combination of purified noribogaine, and kisantin. One formulation comprises a purposefully chosen combination of purified noribogaine, and montanin. One formulation comprises a purposefully chosen combination of purified tabernanthine, and dihydroxyibogamine. One formulation comprises a purposefully chosen combination of purified tabernanthine, and dihydrocatharanthine. One formulation comprises a purposefully chosen combination of purified tabernanthine, and coronaridine. One formulation comprises a purposefully chosen combination of purified tabernanthine, and conopharyngine. One formulation comprises a purposefully chosen combination of purified tabernanthine, and conoflorine. One formulation comprises a purposefully chosen combination of purified tabernanthine, and catharanthine. One formulation comprises a purposefully chosen combination of purified tabernanthine, and iboxygaine. One formulation comprises a purposefully chosen combination of purified tabernanthine, and iboluteine. One formulation comprises a purposefully chosen combination of purified tabernanthine, and ibogamine. One formulation comprises a purposefully chosen combination of purified tabernanthine, and ibogaline. One formulation comprises a purposefully chosen combination of purified tabernanthine, and ibogaine. One formulation comprises a purposefully chosen combination of purified tabernanthine, and epiibogamine. One formulation comprises a purposefully chosen combination of purified tabernanthine, and isovoacangine. One formulation comprises a purposefully chosen combination of purified tabernanthine, and isovoacristine. One formulation comprises a purposefully chosen combination of purified tabernanthine, and kisantin. One formulation comprises a purposefully chosen combination of purified tabernanthine, and montanin. One formulation comprises a purposefully chosen combination of purified tabernanthine, and noribogaine. One formulation comprises a purposefully chosen combination of purified tubotaiwine, and dihydroxyibogamine. One formulation comprises a purposefully chosen combination of purified tubotaiwine, and dihydrocatharanthine. One formulation comprises a purposefully chosen combination of purified tubotaiwine, and coronaridine. One formulation comprises a purposefully chosen combination of purified tubotaiwine, and conopharyngine. One formulation comprises a purposefully chosen combination of purified tubotaiwine, and conoflorine. One formulation comprises a purposefully chosen combination of purified tubotaiwine, and catharanthine. One formulation comprises a purposefully chosen combination of purified tubotaiwine, and iboxygaine. One formulation comprises a purposefully chosen combination of purified tubotaiwine, and iboluteine. One formulation comprises a purposefully chosen combination of purified tubotaiwine, and ibogamine. One formulation comprises a purposefully chosen combination of purified tubotaiwine, and ibogaline. One formulation comprises a purposefully chosen combination of purified tubotaiwine, and ibogaine. One formulation comprises a purposefully chosen combination of purified tubotaiwine, and epiibogamine. One formulation comprises a purposefully chosen combination of purified tubotaiwine, and isovoacangine. One formulation comprises a purposefully chosen combination of purified tubotaiwine, and isovoacristine. One formulation comprises a purposefully chosen combination of purified tubotaiwine, and kisantin. One formulation comprises a purposefully chosen combination of purified tubotaiwine, and montanin. One formulation comprises a purposefully chosen combination of purified tubotaiwine, and noribogaine. One formulation comprises a purposefully chosen combination of purified tubotaiwine, and tabernanthine. One formulation comprises a purposefully chosen combination of purified voacristine, and dihydroxyibogamine. One formulation comprises a purposefully chosen combination of purified voacristine, and dihydrocatharanthine. One formulation comprises a purposefully chosen combination of purified voacristine, and coronaridine. One formulation comprises a purposefully chosen combination of purified voacristine, and conopharyngine. One formulation comprises a purposefully chosen combination of purified voacristine, and conoflorine. One formulation comprises a purposefully chosen combination of purified voacristine, and catharanthine. One formulation comprises a purposefully chosen combination of purified voacristine, and iboxygaine. One formulation comprises a purposefully chosen combination of purified voacristine, and iboluteine. One formulation comprises a purposefully chosen combination of purified voacristine, and ibogamine. One formulation comprises a purposefully chosen combination of purified voacristine, and ibogaline. One formulation comprises a purposefully chosen combination of purified voacristine, and ibogaine. One formulation comprises a purposefully chosen combination of purified voacristine, and epiibogamine. One formulation comprises a purposefully chosen combination of purified voacristine, and isovoacangine. One formulation comprises a purposefully chosen combination of purified voacristine, and isovoacristine. One formulation comprises a purposefully chosen combination of purified voacristine, and kisantin. One formulation comprises a purposefully chosen combination of purified voacristine, and montanin. One formulation comprises a purposefully chosen combination of purified voacristine, and noribogaine. One formulation comprises a purposefully chosen combination of purified voacristine, and tabernanthine. One formulation comprises a purposefully chosen combination of purified voacristine, and tubotaiwine. One formulation comprises a purposefully chosen combination of purified voacangine, and dihydroxyibogamine. One formulation comprises a purposefully chosen combination of purified voacangine, and dihydrocatharanthine. One formulation comprises a purposefully chosen combination of purified voacangine, and coronaridine. One formulation comprises a purposefully chosen combination of purified voacangine, and conopharyngine. One formulation comprises a purposefully chosen combination of purified voacangine, and conoflorine. One formulation comprises a purposefully chosen combination of purified voacangine, and catharanthine. One formulation comprises a purposefully chosen combination of purified voacangine, and iboxygaine. One formulation comprises a purposefully chosen combination of purified voacangine, and iboluteine. One formulation comprises a purposefully chosen combination of purified voacangine, and ibogamine. One formulation comprises a purposefully chosen combination of purified voacangine, and ibogaline. One formulation comprises a purposefully chosen combination of purified voacangine, and ibogaine. One formulation comprises a purposefully chosen combination of purified voacangine, and epiibogamine. One formulation comprises a purposefully chosen combination of purified voacangine, and isovoacangine. One formulation comprises a purposefully chosen combination of purified voacangine, and isovoacristine. One formulation comprises a purposefully chosen combination of purified voacangine, and kisantin. One formulation comprises a purposefully chosen combination of purified voacangine, and montanin. One formulation comprises a purposefully chosen combination of purified voacangine, and noribogaine. One formulation comprises a purposefully chosen combination of purified voacangine, and tabernanthine. One formulation comprises a purposefully chosen combination of purified voacangine, and tubotaiwine. One formulation comprises a purposefully chosen combination of purified voacangine, and voacristine. One formulation comprises a purposefully chosen combination of purified voaluteine, and dihydroxyibogamine. One formulation comprises a purposefully chosen combination of purified voaluteine, and dihydrocatharanthine. One formulation comprises a purposefully chosen combination of purified voaluteine, and coronaridine. One formulation comprises a purposefully chosen combination of purified voaluteine, and conopharyngine. One formulation comprises a purposefully chosen combination of purified voaluteine, and conoflorine. One formulation comprises a purposefully chosen combination of purified voaluteine, and catharanthine. One formulation comprises a purposefully chosen combination of purified voaluteine, and iboxygaine. One formulation comprises a purposefully chosen combination of purified voaluteine, and iboluteine. One formulation comprises a purposefully chosen combination of purified voaluteine, and ibogamine. One formulation comprises a purposefully chosen combination of purified voaluteine, and ibogaline. One formulation comprises a purposefully chosen combination of purified voaluteine, and ibogaine. One formulation comprises a purposefully chosen combination of purified voaluteine, and epiibogamine. One formulation comprises a purposefully chosen combination of purified voaluteine, and isovoacangine. One formulation comprises a purposefully chosen combination of purified voaluteine, and isovoacristine. One formulation comprises a purposefully chosen combination of purified voaluteine, and kisantin. One formulation comprises a purposefully chosen combination of purified voaluteine, and montanin. One formulation comprises a purposefully chosen combination of purified voaluteine, and noribogaine. One formulation comprises a purposefully chosen combination of purified voaluteine, and tabernanthine. One formulation comprises a purposefully chosen combination of purified voaluteine, and tubotaiwine. One formulation comprises a purposefully chosen combination of purified voaluteine, and voacristine. One formulation comprises a purposefully chosen combination of purified voaluteine, and voacangine. One formulation comprises a purposefully chosen combination of purified voacamine, and dihydroxyibogamine. One formulation comprises a purposefully chosen combination of purified voacamine, and dihydrocatharanthine. One formulation comprises a purposefully chosen combination of purified voacamine, and coronaridine. One formulation comprises a purposefully chosen combination of purified voacamine, and conopharyngine. One formulation comprises a purposefully chosen combination of purified voacamine, and conoflorine. One formulation comprises a purposefully chosen combination of purified voacamine, and catharanthine. One formulation comprises a purposefully chosen combination of purified voacamine, and iboxygaine. One formulation comprises a purposefully chosen combination of purified voacamine, and iboluteine. One formulation comprises a purposefully chosen combination of purified voacamine, and ibogamine. One formulation comprises a purposefully chosen combination of purified voacamine, and ibogaline. One formulation comprises a purposefully chosen combination of purified voacamine, and ibogaine. One formulation comprises a purposefully chosen combination of purified voacamine, and epiibogamine. One formulation comprises a purposefully chosen combination of purified voacamine, and isovoacangine. One formulation comprises a purposefully chosen combination of purified voacamine, and isovoacristine. One formulation comprises a purposefully chosen combination of purified voacamine, and kisantin. One formulation comprises a purposefully chosen combination of purified voacamine, and montanin. One formulation comprises a purposefully chosen combination of purified voacamine, and noribogaine. One formulation comprises a purposefully chosen combination of purified voacamine, and tabernanthine. One formulation comprises a purposefully chosen combination of purified voacamine, and tubotaiwine. One formulation comprises a purposefully chosen combination of purified voacamine, and voacristine. One formulation comprises a purposefully chosen combination of purified voacamine, and voacangine. One formulation comprises a purposefully chosen combination of purified voacamine, and voaluteine.

In one embodiment, the disclosed combinations are present within formulations. In one embodiment, the disclosed formulations comprise additional active compounds found within one or more of the following organisms: Tabernaemontana heyneana, Tabernanthe iboga, Ervatamia spp., Tabernaemontana australis, Tabernaemontana divaricata, Tabernaemontana eglandulosa, Tabernaemontana hilariana, Tabernaemontana holstii, Ervatamia orientalis, Tabernaemontana johnstonii, Alstonia scholaris, Peschiera buchtieni, Voacanga africana, and Voacanga schweinfurthii. In one embodiment the disclosed formulations comprise additional active compounds not found within these organisms. In one embodiment the disclosed formulations comprise additional inactive compounds, such as excipients, binders, stabilizers, permeation enhancers, solubilizers, etc.

The formulations disclosed herein are products of human ingenuity, i.e., made by humans and substantially different from how they are found in nature. The disclosed formulations can be distinguished from naturally occurring forms by comparing the cellular pharmacology of the disclosed formulations with that of naturally occurring forms. In one embodiment, the disclosed formulations can be distinguished from naturally occurring forms by comparing the molar ratios of compounds within the disclosed formulations with those found in nature. The disclosed formulations can be distinguished from naturally occurring forms by comparing the molar ratios of compounds within the disclosed formulations with reference compounds that are present alongside the said compounds when those compounds are found in nature.

Disclosed herein is a formulation comprising:

a first purified ibogaine derivative chosen from the group consisting of: dihydroxyibogamine, dihydrocatharanthine, coronaridine, conopharyngine, conoflorine, catharanthine, iboxygaine, iboluteine, ibogamine, ibogaline, ibogaine, epiibogamine, isovoacangine, isovoacristine, kisantin, montanin, noribogaine, tabernanthine, tubotaiwine, voacristine, voacangine, voaluteine, voacamine, 10-hydroxycoronaridine, 10-hydroxyheyneanine, 11-hydroxycoronaridine, 16-demethoxycarbonylvoacamine, 18-methoxycoronaridine, 19-hydroxyvenalstonine, 20-epivoacangarine, 3-(beta-hydroxyethyl)coronaridine, 3-hydroxyvoacangine, 3-oxo-11-methoxytabersonine, 3-oxocoronaridine, affinisine, ajmalicine, akuammicine, akuammidine, albiforanine, angustidine, angustine, angustoline, apparicine, condylocarpine, conoduramine, conodurine, conophyllidine, conophylline, coronaridine hydroxyindolenine, coronaridine pseudoindoxyl, cylindrocarpine, deplancheine, dregamine-1, dregamine-2, echitamidine, echitovenidine, ervadivaricatine B, ervatine, gabunamine, gabunine, geissoschizine, globospiramine, harmaline, harmine, heyneanine hydroxyindolenine, ibogamine-18-carboxylic acid, 12-methoxy-19-oxo-, methyl ester, ibogamine-18-carboxylic acid, 13,20-dihydroxy-, methyl ester, (4alpha,20S)-, isositsirikine, jerantinine A, jerantinine B, jerantinine C, jerantinine D, jerantinine E, jerantinine F, koenimbine, kopsinine, lochnericine, lochneridine, minovin, N-methylibogaine, norfluorocurarine, pericyclivine, perivine, pyrifolidine, pyrifoline, refractine, rhazimine, sarcopharyngine, sewarine, stemmadenine, strictamin, tabernaelegantine A, tabernaelegantine B, tabernaemontanin, tabernamine, tabersonine, tetraphyllicine, tombozine, vallesiachotamine, vinburnine, vincadifformine, vindolinine, vinorine, voacangine hydroxyindolenine, voachalotine, voacorine, voafrine A, voafrine B, voaharine, voaphylline, voaphylline hydroxyindolenine, vobasine, vobtusine, yohimban, (+)20R-15,20-dihydro-cleavamine, (−) 20S-15,20-dihydro-cleavamine, 14S,20R-velbanamine, 20R-1,2-dehydro-pseudo-aspidospermidine, 20S-hydroxy-I,2-dehydro-pseudoaspidospermidine, 19-epi-isovoacristine, 19-acetoxy-11-hydroxytabersonine, 19-hydroxy-11-methoxytabersonine, 19-acetoxy-11-methoxytabersonine, 19-epi-vindolinine, 19(S)-hydroxyibogamine, 19,20-epoxyconoduramine, 19-oxocoronaridine, 3R/S-ethoxy-19-epi-heyneanine, 3R/S-ethoxyheyneanine, desethyl-voacangine, gabonine, hörhammericine, hörhammerinine, iboquine, isovoacryptine, isovocangine, isovoacristine, kisantine, mehranine, pseudotabersonine, pyrifolidine, vinervine, desethyl-voacangine, hystrixnine, 19-oxovoacangine, and loganin and

a second ibogaine derivative selected from the group consisting of: dihydroxyibogamine, dihydrocatharanthine, coronaridine, conopharyngine, conoflorine, catharanthine, iboxygaine, iboluteine, ibogamine, ibogaline, ibogaine, epiibogamine, isovoacangine, isovoacristine, kisantin, montanin, noribogaine, tabernanthine, tubotaiwine, voacristine, voacangine, voaluteine, voacamine, 10-hydroxycoronaridine, 10-hydroxyheyneanine, 11-hydroxycoronaridine, 16-demethoxycarbonylvoacamine, 18-methoxycoronaridine, 19-hydroxyvenalstonine, 20-epivoacangarine, 3-(beta-hydroxyethyl)coronaridine, 3-hydroxyvoacangine, 3-oxo-11-methoxytabersonine, 3-oxocoronaridine, affinisine, ajmalicine, akuammicine, akuammidine, albiforanine, angustidine, angustine, angustoline, apparicine, condylocarpine, conoduramine, conodurine, conophyllidine, conophylline, coronaridine hydroxyindolenine, coronaridine pseudoindoxyl, cylindrocarpine, deplancheine, dregamine-1, dregamine-2, echitamidine, echitovenidine, ervadivaricatine B, ervatine, gabunamine, gabunine, geissoschizine, globospiramine, harmaline, harmine, heyneanine hydroxyindolenine, ibogamine-18-carboxylic acid, 12-methoxy-19-oxo-, methyl ester, ibogamine-18-carboxylic acid, 13,20-dihydroxy-, methyl ester, (4alpha,20S)-, isositsirikine, jerantinine A, jerantinine B, jerantinine C, jerantinine D, jerantinine E, jerantinine F, koenimbine, kopsinine, lochnericine, lochneridine, minovin, N-methylibogaine, norfluorocurarine, pericyclivine, perivine, pyrifolidine, pyrifoline, refractine, rhazimine, sarcopharyngine, sewarine, stemmadenine, strictamin, tabernaelegantine A, tabernaelegantine B, tabernaemontanin, tabernamine, tabersonine, tetraphyllicine, tombozine, vallesiachotamine, vinburnine, vincadifformine, vindolinine, vinorine, voacangine hydroxyindolenine, voachalotine, voacorine, voafrine A, voafrine B, voaharine, voaphylline, voaphylline hydroxyindolenine, vobasine, vobtusine, yohimban, (+)20R-15,20-dihydro-cleavamine, (−) 20S-15,20-dihydro-cleavamine, 14S,20R-velbanamine, 20R-1,2-dehydro-pseudo-aspidospermidine, 20S-hydroxy-I,2-dehydro-pseudoaspidospermidine, 19-epi-isovoacristine, 19-acetoxy-11-hydroxytabersonine, 19-hydroxy-11-methoxytabersonine, 19-acetoxy-11-methoxytabersonine, 19-epi-vindolinine, 19(S)-hydroxyibogamine, 19,20-epoxyconoduramine, 19-oxocoronaridine, 3R/S-ethoxy-19-epi-heyneanine, 3R/S-ethoxyheyneanine, desethyl-voacangine, gabonine, hörhammericine, hörhammerinine, iboquine, isovoacryptine, isovocangine, isovoacristine, kisantine, mehranine, pseudotabersonine, pyrifolidine, vinervine, desethyl-voacangine, hystrixnine, 19-oxovoacangine, and loganin;

wherein the first purified ibogaine derivative is different from the second ibogaine derivative.

A formulation comprising a first purified ibogaine derivative and a second ibogaine derivative additionally comprises a third ibogaine derivative selected from the group consisting of: dihydroxyibogamine, dihydrocatharanthine, coronaridine, conopharyngine, conoflorine, catharanthine, iboxygaine, iboluteine, ibogamine, ibogaline, ibogaine, epiibogamine, isovoacangine, isovoacristine, kisantin, montanin, noribogaine, tabernanthine, tubotaiwine, voacristine, voacangine, voaluteine, voacamine, 10-hydroxycoronaridine, 10-hydroxyheyneanine, 11-hydroxycoronaridine, 16-demethoxycarbonylvoacamine, 18-methoxycoronaridine, 19-hydroxyvenalstonine, 20-epivoacangarine, 3-(beta-hydroxyethyl)coronaridine, 3-hydroxyvoacangine, 3-oxo-11-methoxytabersonine, 3-oxocoronaridine, affinisine, ajmalicine, akuammicine, akuammidine, albiforanine, angustidine, angustine, angustoline, apparicine, condylocarpine, conoduramine, conodurine, conophyllidine, conophylline, coronaridine hydroxyindolenine, coronaridine pseudoindoxyl, cylindrocarpine, deplancheine, dregamine-1, dregamine-2, echitamidine, echitovenidine, ervadivaricatine B, ervatine, gabunamine, gabunine, geissoschizine, globospiramine, harmaline, harmine, heyneanine hydroxyindolenine, ibogamine-18-carboxylic acid, 12-methoxy-19-oxo-, methyl ester, ibogamine-18-carboxylic acid, 13,20-dihydroxy-, methyl ester, (4alpha,20S)-, isositsirikine, jerantinine A, jerantinine B, jerantinine C, jerantinine D, jerantinine E, jerantinine F, koenimbine, kopsinine, lochnericine, lochneridine, minovin, N-methylibogaine, norfluorocurarine, pericyclivine, perivine, pyrifolidine, pyrifoline, refractine, rhazimine, sarcopharyngine, sewarine, stemmadenine, strictamin, tabernaelegantine A, tabernaelegantine B, tabernaemontanin, tabernamine, tabersonine, tetraphyllicine, tombozine, vallesiachotamine, vinburnine, vincadifformine, vindolinine, vinorine, voacangine hydroxyindolenine, voachalotine, voacorine, voafrine A, voafrine B, voaharine, voaphylline, voaphylline hydroxyindolenine, vobasine, vobtusine, yohimban, (+)20R-15,20-dihydro-cleavamine, (−) 20S-15,20-dihydro-cleavamine, 14S,20R-velbanamine, 20R-1,2-dehydro-pseudo-aspidospermidine, 20S-hydroxy-1,2-dehydro-pseudoaspidospermidine, 19-epi-isovoacristine, 19-acetoxy-11-hydroxytabersonine, 19-hydroxy-11-methoxytabersonine, 19-acetoxy-11-methoxytabersonine, 19-epi-vindolinine, 19(S)-hydroxyibogamine, 19,20-epoxyconoduramine, 19-oxocoronaridine, 3R/S-ethoxy-19-epi-heyneanine, 3R/S-ethoxyheyneanine, desethyl-voacangine, gabonine, hörhammericine, hörhammerinine, iboquine, isovoacryptine, isovocangine, isovoacristine, kisantine, mehranine, pseudotabersonine, pyrifolidine, vinervine, desethyl-voacangine, hystrixnine, 19-oxovoacangine, and loganin;

wherein each of the first purified ibogaine derivative, the second ibogaine derivative, and the third ibogaine derivative are structurally distinct from one another.

The disclosed formulations may by prepared by purifying one or more ibogaine derivatives and then specifically recombining a particular amount of a first purified ibogaine derivative with a particular amount of one or more second ibogaine derivatives. Accordingly, the formulations disclosed herein comprise a combination of a particular amount of first purified ibogaine derivative and a particular amount of a second ibogaine derivative. The formulations disclosed herein comprise a combination of a first purified ibogaine derivative and a second purified ibogaine derivative. The formulations disclosed herein may also comprise a combination of a first purified ibogaine derivative, a second purified ibogaine derivative, and a third purified ibogaine derivative.

At the time of this disclosure, the disclosed combinations of ibogaine derivatives were only available within complex mixtures comprising other plant matter. Accordingly, each of the combinations disclosed herein differs from previously known compositions in many different ways. For example, in the formulations disclosed herein, the ratio of ibogaine derivatives (e.g., the first purified ibogaine derivatives to the second ibogaine derivative) or the ratio of an ibogaine derivative to a naturally occurring reference compound (e.g., cellulose, ligin, chlorophyll, etc) differs from those previously disclosed or otherwise naturally occurring.

Disclosed herein are methods of regulating the activity of a neurotransmitter receptor by administering a first dosage formulation comprising a first purified ibogaine derivative to the said receptor. In one embodiment, the methods and compositions disclosed herein comprise administering a first dosage formulation comprising a second ibogaine derivative. In one embodiment, the methods disclosed herein comprise administering a first dosage formulation comprising a first purified cannabinoid. In one embodiment, the methods disclosed herein comprise administering a first dosage formulation comprising a first purified terpene. In one embodiment, the methods disclosed herein comprise administering a first dosage formulation comprising a neurotransmitter activity modulator. In one embodiment, the methods disclosed herein comprise administering a first dosage formulation comprising a first purified ibogaine derivative, a first purified cannabinoid, a first purified terpene, and/or a neurotransmitter activity modulator.

In one embodiment, the methods disclosed herein comprise administering one or more of the formulations disclosed herein. In one embodiment, the methods disclosed herein comprise treating a psychological disorder, e.g., an anxiety disorder, a compulsive disorder (e.g., an addiction), a depressive disorder, etc., with the compositions disclosed herein, e.g., a composition with one or more ibogaine derivatives, a composition with one or more cannabinoids, a composition with one or more terpenes, and/or a combination thereof. In one embodiment, the methods disclosed herein comprise treating a psychological disorder, e.g., an anxiety disorder, a compulsive disorder (e.g., and addiction), a depressive disorder, etc., by administering to a subject in need of treatment one or more of the compositions disclosed herein and a neurotransmitter activity modulator, e.g., a serotonergic drug, a dopaminergic drug, etc.

Identifying a person in need of treatment for depression or addiction can be done by applying the criteria that are set forth in the Diagnostic and Statistical Manual of Mental Disorders (DSM-5).

In one embodiment, the combinations disclosed herein are present within formulations made by humans. Such formulations are the product of human ingenuity as opposed to combinations of molecules that are substantially unaltered from the forms in which they are found in nature. Accordingly, formulations disclosed herein include combinations of ibogaine derivatives which differ substantially from how those combinations of ibogaine derivatives are found within nature. Such differences can be appreciated by measuring the physical properties of the formulations, e.g., by comparing the ratio of the components within the formulations disclosed herein with the ratio of those components within naturally occurring combinations, such as plant matter or plant extracts. Plants and plant extracts often do not provide the same physical, cellular and/or clinical properties as formulations made by combining particular compounds of known purity. This distinction can be readily ascertained by conducting experiments well known in the art, such as cellular binding studies, clinical trials, and other chemical and biological assays designed for measuring particular properties of molecules and combinations thereof.

Disclosed herein are new formulations comprising a combination of a first purified ibogaine derivative and a serotonergic drug. In one embodiment, the compositions disclosed herein comprise a first purified ibogaine derivative and a serotonergic drug present in purposefully engineered and unnaturally occurring molar ratios, such as those described above for the combination of ibogaine derivatives.

Disclosed herein are new formulations comprising a combination of a first purified ibogaine derivative and a first purified cannabinoid. In one embodiment, the compositions disclosed herein comprise a first purified ibogaine derivative and a first purified cannabinoid present in purposefully engineered and unnaturally occurring molar ratios, such as those described above for the combination of ibogaine derivatives.

Disclosed herein are new formulations comprising a combination of a first purified ibogaine derivative and a first purified psilocybin derivative, present in purposefully engineered and unnaturally occurring molar ratios, such as those described above for the combination of ibogaine derivatives.

Disclosed herein are new formulations comprising a combination of a first purified ibogaine derivative and a first purified terpene. In one embodiment, the compositions disclosed herein comprise a first purified ibogaine derivative and a first purified terpene present in purposefully engineered and unnaturally occurring molar ratios, such as those described above for the combination of ibogaine derivatives.

Disclosed herein are new formulations comprising a combination of a first purified ibogaine derivative, a first purified cannabinoid, and a first purified terpene. In one embodiment, the compositions disclosed herein comprise a first purified ibogaine derivative, a first purified cannabinoid, and a first purified terpene present in purposefully engineered and unnaturally occurring molar ratios, such as those described above for the combination of ibogaine derivatives.

Disclosed herein are new formulations comprising a combination of a first purified ibogaine derivative, a first purified cannabinoid, and a second purified cannabinoid. In one embodiment, the compositions disclosed herein comprise a first purified ibogaine derivative, a first purified cannabinoid, and a second purified cannabinoid present in purposefully engineered and unnaturally occurring molar ratios, such as those described above for the combination of ibogaine derivatives.

In one example, for a 25 g composition comprising 2 g of an ibogaine derivative. The amount of an ibogaine derivative within a composition is determined by the following:

2.0 g of ibogaine derivative÷25.0 g of composition×100%=8.0%

Disclosed herein is a method of treating tumors comprising administering one or more purified ibogaine derivatives chosen from catharanthine, coronaridine, isovoacangine, voacristine, and voacangine.

Disclosed herein is a method of treating pain comprising administering one or more ibogaine derivatives chosen from conopharyngine or tubotaiwine.

Disclosed herein is a method of treating addiction comprising administering the ibogaine derivative ibogamine.

Disclosed herein is a method of treating Alzheimer's disease and vascular dementia comprising administering the purified ibogaine derivative coronaridine.

Disclosed herein is a method of treating convulsions comprising administering one or more ibogaine derivatives chosen from ibogamine and montanine.

Disclosed herein is a method of treating anxiety comprising administering the purified ibogaine derivative montanine.

Disclosed herein is a method of treating depression comprising administering the ibogaine derivative montanine.

Disclosed herein are compositions comprising two or more ibogaine derivatives and substantially free from a particular ibogaine derivative that naturally co-occurs with the two or more ibogaine derivatives within their natural source, e.g., iboga.

In one embodiment, the composition comprises an iboga extract that is substantially free from ibogaine. In one embodiment, the composition comprises an iboga extract that is substantially free from dihydroxyibogamine. In one embodiment, the composition comprises an iboga extract that is substantially free from dihydrocatharanthine. In one embodiment, the composition comprises an iboga extract that is substantially free from coronaridine. In one embodiment, the composition comprises an iboga extract that is substantially free from conopharyngine. In one embodiment, the composition comprises an iboga extract that is substantially free from conoflorine. In one embodiment, the composition comprises an iboga extract that is substantially free from catharanthine. In one embodiment, the composition comprises an iboga extract that is substantially free from iboxygaine. In one embodiment, the composition comprises an iboga extract that is substantially free from iboluteine. In one embodiment, the composition comprises an iboga extract that is substantially free from ibogamine. In one embodiment, the composition comprises an iboga extract that is substantially free from ibogaline. In one embodiment, the composition comprises an iboga extract that is substantially free from epiibogamine. In one embodiment, the composition comprises an iboga extract that is substantially free from isovocangine. In one embodiment, the composition comprises an iboga extract that is substantially free from isovoacristine. In one embodiment, the composition comprises an iboga extract that is substantially free from kisantin. In one embodiment, the composition comprises an iboga extract that is substantially free from montanin. In one embodiment, the composition comprises an iboga extract that is substantially free from noribogaine. In one embodiment, the composition comprises an iboga extract that is substantially free from tabernanthine. In one embodiment, the composition comprises an iboga extract that is substantially free from tubotaiwine. In one embodiment, the composition comprises an iboga extract that is substantially free from voacristine. In one embodiment, the composition comprises an iboga extract that is substantially free from voacangine. In one embodiment, the composition comprises an iboga extract that is substantially free from voaluteine. In one embodiment, the composition comprises an iboga extract that is substantially free from voacamine.

Iboga extracts can be made according to a variety of methods that are known in the art. For example, in 2019, Krengel et al. published a study using gas chromatography/mass spectrometry (GC/MS) to examine the efficiency of methanol, acetone, ethyl acetate, dichloromethane, chloroform, and hydrochloric acid for extracting individual compounds from ibogaine-containing species.³⁷

Individual compounds can be removed from an iboga extract through a variety of methods that are known in the art. For example, in 1958, Dickel et al. published several methods for isolating and removing single compounds from whole iboga plant extracts.³⁸

Within the context of this disclosure, the term “purified” means separated from other materials, such as plant or fungal material, e.g., protein, chitin, cellulose, or water. In one embodiment, the term “purified” refers to a compound substantially free of other materials. In one embodiment, the term “purified” refers to a compound that is substantially free from a second tryptamine compound. In one embodiment, the term “purified” refers to a compound substantially free from histidine. In one embodiment, the term “purified” refers to a compound substantially free from a biological material, such as mold, fungus, plant matter, or bacteria. In one embodiment, the term “purified” refers to a compound substantially free from a different unwanted compound, e.g., a compound correlated with unwanted side effects.

Other examples of unpurified ibogaine derivatives would include organisms containing ibogaine derivatives and/or naturally occurring material such as biological material and/or structural material such as cellulose or chitin. Examples of those organisms include Tabernaemontana heyneana, Tabernanthe iboga, Ervatamia spp., Tabernaemontana australis, Tabernaemontana divaricata, Tabernaemontana eglandulosa, Tabernaemontana hilariana, Tabernaemontana holstii, Ervatamia orientalis, Tabernaemontana johnstonii, Alstonia scholaris, Peschiera buchtieni, Voacanga africana, and Voacanga schweinfurthii. Similarly, the term “cannabis extract” or “cannabinoid extract” traditionally refers to whole plant (aka crude or full spectrum extracts) which have not been subjected to further purification to eliminate unwanted molecules that naturally occur in the cannabis plant. For example, a “cannabis extract comprising cannabidiol” could be expected to include cannabidiol (aka “CBD”) and also varying amounts of other compounds, including cannabinoids, terpenes, and other biological material.

In one embodiment, the term “purified” refers to a compound or composition that has been crystallized.

In one embodiment, the term “purified” refers to a compound or composition that has been chromatographed, for example by gas chromatography, liquid chromatography (e.g., LC, H PLC, etc.), flush column chromatography, etc.

In one embodiment, the term “purified” refers to a compound or composition that has been distilled.

In one embodiment, the term “purified” refers to a compound or composition that has been sublimed.

In one embodiment, the term “purified” refers to a compound or composition that has been subject to two or more steps chosen from crystallization, chromatography, distillation, or sublimation.

In one embodiment, the term “purified” refers to a compound that is between 80-100% pure, meaning that the compound makes up 80-100% of the total mass of the composition.

In one embodiment, the term “purified” refers to a compound that is between 90-100% pure, meaning that the compound makes up 90-100% of the total mass of the composition.

In one embodiment, the term “purified” refers to a compound that is between 95-100% pure, meaning that the compound makes up 95-100% of the total mass of the composition.

In one embodiment, the term “purified” refers to a compound that is between 99-100% pure, meaning that the compound makes up 99-100% of the total mass of the composition.

In one embodiment, the term “purified” refers to a compound that is between 99.9-100% pure, meaning that the compound makes up 99.9-100% of the total mass of the composition.

GC/MS can be used for identification and quantification of one or more ibogaine derivatives in biological specimens.

Ibogaine derivatives can also be identified by using liquid chromatography with or without tandem mass spectrometry (LC/MS/MS). When using LC/MS, method derivatization prior to analysis is often not required. Liquid chromatography combined with tandem mass spectrometry for analysis of psilocin and psilocybin in psychoactive mushrooms may also provide useful samples. All data indicate that the presence and amounts of psychoactive compounds within naturally occurring samples is considered to be highly variable. Accordingly, this disclosure provides formulations made with known amounts of known compounds, including known amounts of ibogaine derivatives. Such formulations allow for administering consistent amounts of ibogaine derivatives, which provides the user or subject with consistent and reliable effects.

The effectiveness of drugs on treating depressive symptoms can be experimentally determined by using animals and human clinical trials that are known in the art. For example, the forced swim test (FST) measures depressive behavior in rodents.³⁹ The animals are placed in an inescapable tank filled with water. It is theorized that depressed animals will make less of an effort to escape from the tank than those not depressed. Measuring their escape-related mobility behavior quantifies the antidepressive effects of the drug/treatment.

According to the American Psychological Association (APA), there are several “instruments” for measuring depressive symptoms and the efficacy of treatment in humans. These include the Beck Depression Inventory, Hamilton Depression Rating Scale, Beck Hopelessness Scale, and the Quick Inventory of Depressive Symptomology-Self Report.

The effectiveness of drugs on treating addiction can be experimentally determined by using animals and human clinical trials that are known in the art. For example, in 2015, Chang et al. trained rats to self-administer nicotine intravenously prior to testing the effects of noribogaine on nicotine addiction.⁴⁰ The National Institute on Drug Abuse (NIDA) provides several tools for screening and assessing alcohol and drug addiction in patients. These tools include the Alcohol Use Disorders Identification Test, Opioid Risk Tool, Drug Abuse Screen Test, and the NIDA Drug Use Screening Tool.

In one embodiment, the term “substantially free” means less than 10% of the molar concentration of the first ibogaine derivative. In one embodiment, the term “substantially free” means less than 5% of the molar concentration of the first ibogaine derivative. In one embodiment, the term “substantially free” means less than 2% of the molar concentration of the first ibogaine derivative. In one embodiment, the term “substantially free” means less than 1% of the molar concentration of the first ibogaine derivative. In one embodiment, the term “substantially free” means less than 0.5% of the molar concentration of the first ibogaine derivative. In one embodiment, the term “substantially free” means less than 0.1% of the molar concentration of the first ibogaine derivative.

Such molar concentrations can be determined by methods known in the art, including, but not limited to, chromatography, mass spectroscopy, nuclear magnetic resonance spectroscopy (NMR), etc.

1. A formulation comprising a combination comprising a first purified ibogaine derivative and a second ibogaine derivative.

2. The formulation of claim 1, wherein the combination comprises the first purified ibogaine derivative and a second purified ibogaine derivative.

3. The formulation of claim 1, wherein the combination comprises the first purified ibogaine derivative, a second purified ibogaine derivative, and a third ibogaine derivative.

4. The formulation of claim 1, wherein the combination comprises the first purified ibogaine derivative, a second purified ibogaine derivative, and a third purified ibogaine derivative.

5. The formulation of claim 1, wherein the first purified ibogaine derivative is selected from the group consisting of dihydroxyibogamine, dihydrocatharanthine, coronaridine, conopharyngine, conoflorine, catharanthine, iboxygaine, iboluteine, ibogamine, ibogaline, ibogaine, epiibogamine, isovoacangine, isovoacristine, kisantin, montanin, noribogaine, tabernanthine, tubotaiwine, voacristine, voacangine, voaluteine, and voacamine.

6. The formulation of claim 1, wherein the combination is substantially free from a compound selected from the group consisting of dihydroxyibogamine, dihydrocatharanthine, coronaridine, conopharyngine, conoflorine, catharanthine, iboxygaine, iboluteine, ibogamine, ibogaline, ibogaine, epiibogamine, isovoacangine, isovoacristine, kisantin, montanin, noribogaine, tabernanthine, tubotaiwine, voacristine, voacangine, voaluteine, and voacamine.

7. The formulation of claim 6, wherein the combination is substantially free from ibogaine.

8. A method of treating addiction in a human in need of treatment, comprising the step of administering a therapeutically effective amount of a formulation of claim 1 to the human in need of treatment.

9. The method of claim 8, wherein the addiction is selected from the group consisting of opioid addiction, alcohol addiction, and nicotine addiction.

10. The method of claim 9, wherein the addiction is opioid addiction.

11. A method of treating depression in a human in need of treatment, comprising the step of administering a therapeutically effective amount of a formulation of claim 1 to the human in need of treatment.

12. The method of claim 11, wherein the depression is selected from the group consisting of a major depressive disorder and treatment resistant depression.

13. The method of claim 11, comprising administering ibogaine and noribogaine.

14. The method of claim 12, comprising administering a composition comprising purified ibogaine.

15. The method of claim 11, comprising administering a composition comprising purified noribogaine.

16. The method of claim 8, comprising administering ibogaine and noribogaine to the brain of the human in need of treatment.

17. The method of claim 8, comprising administering a composition of claim 1 intravenously to the human in need of treatment.

18. The method of claim 8, comprising administering between 1 to 100 mg/kg of total ibogaine derivatives to the human in need of treatment.

19. The method of claim 18, comprising administering between 2 to 25 mg/kg of total ibogaine derivatives to the human in need of treatment.

20. The method of claim 16, comprising administering a composition comprising purified ibogaine and purified noribogaine to the brain of the human in need of treatment.
